(19) United States
(12) Patent Application Publication
(10) Pub. No.: US 2019/0213910 A1
(43) Pub. Date: Jul. 11, 2019

(54) METHOD AND SYSTEM FOR FOOD, BEVERAGE, OR MEDICINE TRACKING AND CONSUMPTION THRESHOLDS

(71) Applicant: KONINKLIJKE PHILIPS N.V., EINDHOVEN (NL)

(72) Inventors: LAURENTIA JOHANNA HUIJBREGTS, EINDHOVEN (NL); RICK BEZEMER, AMSTERDAM (NL)

(73) Assignee: KONINKLIJKE PHILIPS N.V., EINDHOVEN (NL)

(21) Appl. No.: 16/326,558

(22) PCT Filed: Aug. 23, 2017
(86) PCT No.: PCT/EP2017/071176

§ 371 Date: Feb. 19, 2019

Related U.S. Application Data
(60) Provisional application No. 62/378,265, filed on Aug. 23, 2016.

(51) Int. Cl. G09B 19/00; G16H 20/60

(52) U.S. Cl. CPC G09B 19/00; G16H 20/60

(57) ABSTRACT

A method, system, and device for providing a consumption threshold to a user regarding consumption of an item such as coffee. The method comprises the steps of: receiving (420) one or more goals from the user; generating (430), based on the received one or more goals from the user, a consumption threshold; providing (440) the generated consumption threshold to the user; receiving (450) information about the user; updating (460), based on the information about the user, the consumption threshold; and providing (440) the updated consumption threshold to the user.

### TECHNICAL FIELD

The present disclosure is directed generally to methods and systems for tracking food and beverage consumption and providing intake recommendations, and more specifically, but not exclusively, to methods and systems for personalized tracking of coffee consumption using variable intake thresholds.

### BACKGROUND

Coffee beans are one of the most commonly traded commodities in the world, and coffee is one of the most popular drinks in the world. Across the globe millions of people consume at least one cup of coffee every day, and many millions of those people consume multiple cups of coffee a day.

Coffee is typically consumed for the stimulating effect resulting from the caffeine extracted from the coffee beans. Although clinical studies have suggested that moderate coffee consumption is either benign or mildly beneficial to healthy adults, the substances in coffee can affect different people in different ways. Often, depending on the amount of coffee consumed together with the consumer's personal physiological response, coffee can have either beneficial or harmful effects. Indeed, one individual may react negatively to the same amount of coffee that another individual can consume without effect. Since coffee can increase the consumer's alertness and/or activity and can suppress appetite, it may help in losing weight. In contrast, too much coffee can lead to stress and increase sleep onset latency, and in some individuals can even result in serious health consequences such as arrhythmias or migraines.

Although individual responses to coffee vary widely, guidelines for coffee consumption are typically based on entire populations and therefore do not take into account the individual responses of consumers. Guidelines also do not account for the different goals that individuals who consumer coffee may possess, including lowering blood pressure, avoiding arrhythmias, staying active, improving sleep, getting rid of coffee addiction, and/or losing weight, among others.

### SUMMARY

In view of the foregoing, it would be beneficial to provide methods and systems that provide individualized consumption thresholds for food, beverage, and/or medicine consumption.

The present disclosure is directed to methods and systems for providing individualized recommendations for food, beverage, and/or medicine consumption. Various embodiments and implementations herein are directed to a device or system that tracks consumption of an item, such as coffee, and generates recommendations and consumption thresholds that are delivered to the user. The system can consider goals such as weight loss, migraine avoidance, sleep efficiency, and other goals when generating the recommendations and consumption thresholds. The consumption thresholds will change over time, including over the course of a 24-hour period, based on tracked consumption, user goals, and other factors, including but not limited to activity levels, consumption of other items that affect the tracked item, and others. The consumption threshold can be a minimum threshold, a maximum threshold, and/or an optimal threshold, among other possible thresholds.

Generally, in one aspect, a computer-implemented method for providing a recommendation to a user about consumption of an item is provided. The method includes the steps of: (i) receiving one or more goals from the user; generating, based on the received one or more goals from the user, a consumption threshold; (ii) providing the generated consumption threshold to the user; (iii) receiving information about the user; (iv) updating, based on the information about the user, the consumption threshold; and (v) providing the updated consumption threshold to the user.

According to an embodiment, the consumption threshold is updated a plurality of times during a 24-hour period.

According to an embodiment, the consumption threshold comprises a minimum consumption threshold, a maximum consumption threshold, and/or an optimal consumption threshold.

According to an embodiment, the information about the user comprises the amount of the item consumed and the time of consumption.

According to an embodiment, the updating step is based at least in part on an activity level of the item by the user.

According to an embodiment, the updating step is based at least in part on an estimated amount of the item in the user's body.

According to an embodiment, the method further includes the step of receiving physiological data about the user, and the updating step is based at least in part on the received physiological data, wherein physiological data comprises one or more of physical data, behavioral data, emotional data, and/or one or more other types of data.

According to an embodiment, the physiological data about the user comprises one or more of blood pressure, heart rate, pulse, blood oxygen, body temperature, respiratory rate, and activity level.

According to an embodiment, the physiological data is received from a sensor of a wearable device.

According to an embodiment, the information about the user is received from a wearable device or a consumption monitoring device.

According to an embodiment, the method further includes the step of notifying the user if a level of the item is below the minimum threshold or above the maximum threshold.

According to an embodiment, the step of generating a consumption threshold comprises the steps of (i) analyzing at least two goals provided by the user, the at least two goals resulting in at least two different consumption thresholds, and (ii) selecting one of the at least two different consumption thresholds.

According to an embodiment, the item is a consumable that affects the cardiovascular system, such as coffee.

According to an aspect is a device for providing an item consumption threshold to a user. The device includes: a processor configured to: (i) receive one or more goals from the user; (ii) generate, based on the received one or more goals from the user, a consumption threshold; (iii) receive information about the user; and update, based on the information about the user, the consumption threshold; and a display configured to provide the generated consumption threshold and updated consumption threshold to the user.

According to an aspect is a system for providing an item consumption threshold to a user. The system includes: a consumption monitoring device configured to generate information about the user's consumption of the item, wherein the generated information is communicated to a processor, wherein the processor is configured to: (i) receive one or more goals from the user; (ii) generate, based on the received one or more goals from the user, a maximum and/or minimum consumption threshold; (iii) receive information about the user's consumption of the item; and update, based on the information about the user's consumption of the item, the consumption threshold; and a display configured to provide the generated consumption threshold and updated consumption threshold to the user.

According to an aspect is a computer-implemented method for providing a recommendation to a user about consumption of an item is provided. The method includes the steps of: (i) receiving one or more goals from the user; generating, based on the received one or more goals from the user, a consumption threshold; (ii) providing the generated consumption threshold to the user; (iii) receiving information about the user; (iv) updating, based on the information about the user, the consumption threshold; and (v) providing the updated consumption threshold to the user.

According to an embodiment, the information about the user is physiological information about the user, and/or information about an activity of the user.

According to an embodiment, the step of updating the consumption threshold comprises the step of prioritizing the received one or more goals from the user, and the updating step utilizes the prioritized goals to update the consumption threshold.

As used herein for purposes of the present disclosure, the term “processor” is used generally to describe various apparatus relating to the operation of the recommendation apparatus, system, or method. A processor can be implemented in numerous ways (e.g., such as with dedicated hardware) to perform various functions discussed herein. A “processor” can employ one or more microprocessors that may be programmed using software (e.g., microcode) to perform various functions discussed herein. A processor may also be implemented as a combination of dedicated hardware to perform some functions. Examples of processor components that may be employed in various embodiments of the present disclosure include, but are not limited to, conventional microprocessors, application specific integrated circuits (ASICs), and field-programmable gate arrays (FPGAs).

In various implementations, a processor may be associated with one or more storage media (generically referred to herein as “memory,” e.g., volatile and non-volatile computer memory such as RAM, PROM, EPROM, and EEPROM, floppy disks, compact disks, optical disks, magnetic tape, etc.). In some implementations, the storage media may be encoded with one or more programs that, when executed on one or more processors and/or controllers, perform at least some of the functions discussed herein. Various storage media may be fixed within a processor or controller or may be transportable, such that the one or more programs stored thereon can be loaded into a processor or controller so as to implement various aspects discussed herein. The terms “program” or “computer program” are used herein in a generic sense to refer to any type of computer code (e.g., software or microcode) that can be employed to program one or more processors or controllers. As used herein, the term “non-transitory machine-readable medium” will be understood to encompass both volatile and non-volatile memories, but to exclude transitory signals.

The term “user interface” as used herein refers to an interface between a human user or operator and one or more devices that enables communication between the user and the device(s). Examples of user interfaces that may be employed in various implementations of the present disclosure include, but are not limited to, switches, potentiometers, buttons, dials, sliders, track balls, display screens, various types of graphical user interfaces (GUIs), touch screens, microphones and other types of sensors that may receive some form of human-generated stimulus and generate a signal in response thereto.

Various embodiments of the present invention may further include non-transitory computer-readable storage media, having embodied thereon a firewall program executable by a processor to perform methods described herein.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein.

These and other aspects will be apparent from and elucidated with reference to the embodiment(s) described hereinafter.

### BRIEF DESCRIPTION OF THE DRAWINGS

In the drawings, like reference characters generally refer to the same parts throughout the different views. Also, the drawings are not necessarily to scale, emphasis instead generally being placed upon illustrating the principles of the disclosure.

FIG. 1 is a block diagram of a system for providing individualized recommendations, in accordance with an embodiment.

FIG. 2 is a block diagram of a system for providing individualized recommendations, in accordance with an embodiment.

FIG. 3 is a block diagram of a system for providing individualized recommendations, in accordance with an embodiment.

FIG. 4 is a flowchart of a method for providing consumption thresholds, in accordance with an embodiment.

FIG. 5 is a flowchart of a method for providing consumption thresholds, in accordance with an embodiment.

FIG. 6 is a timeline of coffee consumption and consumption thresholds, in accordance with an embodiment.

FIG. 7 is a subset of the minimum consumption threshold of FIG. 5, in accordance with an embodiment.

### DETAILED DESCRIPTION OF EMBODIMENTS

The present disclosure is directed to methods and systems for providing individualized recommendations for food, beverage, and/or medicine consumption. More generally, Applicant has recognized and appreciated that it would be beneficial to provide a device or system that tracks consumption of an item, such as coffee, and generates recommendations and consumption thresholds that are delivered to the user. A particular goal of utilization of certain embodiments of the present disclosure is to provide variable consumption recommendations and thresholds to a coffee consumer that change over time to account for current consumption and one or more goals set by that consumer.

In view of the foregoing, various embodiments and implementations are directed to a system that receives coffee consumption data about a consumer, and one or more personal goals set by that consumer. The data about coffee consumption may be collected, for example, via a wearable, coffee machine, and/or intelligent coffee cup, among other sources, including from the user via a manual input. The received data and goals are analyzed to determine consumption thresholds and/or recommendations for the consumer.

In addition to coffee consumption, various embodiments and implementations herein are directed to a system or method that utilizes physiological data and consumption data about a consumable other than coffee, including any consumed beverage or food, medication, treatment, remedy, or other consumable. Notably, a consumable can be an item that is taken by the user via a method other than eating or drinking. For example, the consumed item could be an inhalable such as a gas or vaporized item. As another example, the consumed item could be an injectable such as a liquid that is injected into the individual. In particular, the system or method could relate to a consumable that may affect the cardiovascular system of the consumer.

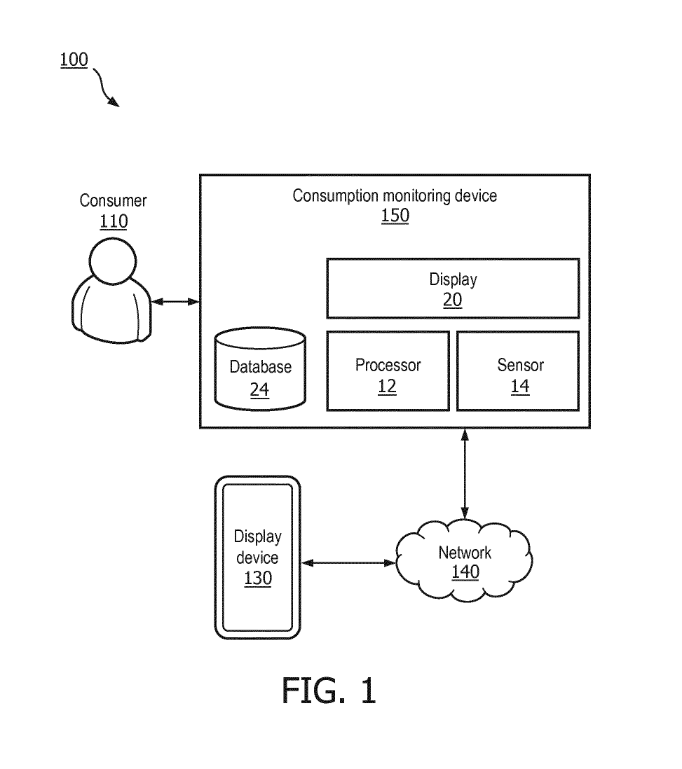

Referring to FIG. 1, in one embodiment, is a system 100 for providing individualized recommendations and/or thresholds for coffee consumption. System 100 comprises a consumer 110 and a consumption monitoring device 150. The system also optionally includes a network 140 and a display device 130. Notably, while the system comprises hardware, the system also comprises software components. For example, at least a portion of the personalized consumption recommendation system may be an application installed and running on a smartphone, computer, tablet, or other computerized device. Additionally, at least a portion of the personalized consumption recommendation system may be software as a service, hosted in the cloud or locally on a server or computer, which receives information from the consumer. It analyzes the information and then provides recommendations to the consumer, such as part of a subscription service.

The consumer 110 is any individual that has consumed or is planning to consume a beverage, food, and/or medicine that may affect the user physiologically. For example, the individual may be an average consumer of coffee, an above-average consumer of coffee, or an individual interested in beginning to consume coffee. The individual may also be a consumer of any other beverage, food, or medicine that the consumer wishes to track.

According to this embodiment, the consumption monitoring device 150 collects information about the consumer's consumption of the beverage. Accordingly, the device may comprise, for example, one or more of a processor 12, sensor 14, user interface 16, communications module 18, display 20, memory 22, database 24, and/or battery or power supply 26. The processor 12 may be programmed using software to perform various functions discussed herein, and can be utilized in combination with a memory 22 and/or database 24. Memory 22 and/or database 24 can store data, including one or more commands or software programs for execution by processor 12, as well as various types of data. For example, the memory 22 may comprise a non-transitory computer readable storage medium that includes a set of instructions that are executable by processor 12, and which cause the system to execute one or more of the steps of the methods described herein. The user interface may be a button or multiple buttons, a microphone, a key stroke input, or any of a variety of other inputs. According to another embodiment, consumer 110 may provide the input remotely via a computer which communicates the input to the wearable device via network 140.

The consumption monitoring device 150 can be, for example, any device that monitors or provides information about the consumer's consumption of the beverage of interest. For example, consumption monitoring device 150 may be a coffee maker. The coffee maker can track coffee made and/or consumed by the consumer. For example, the coffee maker may comprise a volume sensor, a pour sensor, a motion sensor, or any of a variety of other monitors or sensors to obtain information about coffee creation and/or consumption, or it could store or transmit information about which button(s) has been pressed to create the coffee, which in some coffee machines automatically reveals the strength and amount of coffee. According to another embodiment, consumption monitoring device 150 may be an intelligent handheld device associated with beverage consumption, such as a bottle, cup, mug, or thermos. The cup could comprise, for example, temperature, motion, pressure, capacitance, and/or other sensors to obtain information about beverage consumption by the consumer.

The consumption monitoring device 150 can also comprise one or more sensors 14 configured to obtain physiological information about the consumer. For example, the device may comprise electrodes to obtain heart rate, a camera to obtain one or more vital signs such as blood pressure, pulse, and breathing rate, and/or any of a variety of other sensors.

According to an embodiment, the consumption monitoring device 150 may also comprise a wired and/or wireless communications module to communicate information, optionally via network 140, to another device, such as computing device 130. The communications module can facilitate communication with one or more networks or with other devices, for example, by using any wired and/or wireless methods that are known, including but not limited to Wi-Fi, Bluetooth, 3G, 4G, LTE, and/or ZigBee, among others. The computing device will include, for example, a display that provides information to the consumer as described or otherwise envisioned herein. This enables the consumer to receive the information, which may include the personalized recommendations, while located remotely from the consumption monitoring device 150.

In some embodiments, the coffee consumption monitoring device 150 may not be a dedicated device and may instead be incorporated into, for example, the wearable device 120 or a mobile device (e.g., a phone or tablet) of the consumer 110. The coffee consumption monitoring device 150 may enable the consumer 110 to manually identify coffee consumption events or may identify such events using contextual information such as location (e.g., near a coffee machine or in a coffee shop), temperature changes (e.g., near the consumer's hand when the user picks up a coffee cup), hand motions (e.g., those indicative of drinking), time of day, consumer habit information, physiological sensor data, etc. In some embodiments, a trained model (e.g., a classifier) may be used to use multiple types of information to draw inferences about coffee consumption events.

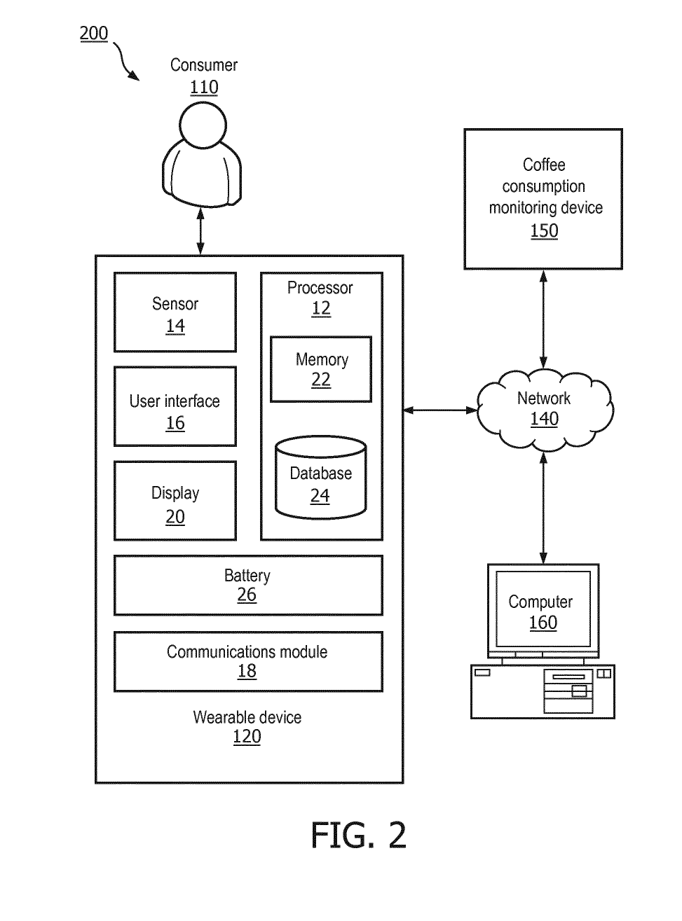

Referring to FIG. 2, in one embodiment, is a system 200 for providing individualized recommendations and/or thresholds for coffee consumption. System 200 comprises a consumer 110, a wearable device 120, and a coffee consumption monitoring device 150. The system also optionally includes a network 140 and a computer or server 160.

Wearable device 120 may be any device suitable for collecting the information utilized in the methods described or otherwise envisioned herein. According to an embodiment, the wearable device 120 may be any type of mobile electronic device that can be worn on the body, any device that is attached to or embedded in clothes, and various other accessories of an individual. Examples of some wearable technology include wrist-worn (e.g., wristwatch) devices or adhesive patches that include sensor devices such as accelerometers, PPG sensors, ECG sensors, etc. According to some embodiments, the wearable device 120 is a smartphone or similar mobile computing device. The wearable device comprises a processor 12 which is configured or programmed to receive one or more signals from one or more sensors 14, a user interface 16, and/or a communications module 18, and configured or programmed to transmit one or more signals to a display 20.

According to one embodiment, the wearable device 120 is worn on or near one or more ears of the consumer. For example, the wearable device 120 may be one or two earbuds worn by the user, or may be integrated into a headset. As another example, the wearable device may be an earpiece worn at least partially behind the ear. This positioning of the wearable device will be inconspicuous, but will also enable the collection of accurate physiological data.

The wearable device also comprises a battery or power supply 26, which provides power for operation of the wearable device 120. The battery or power supply 26 may be implemented through the use of a lithium ion battery, for example. In some embodiments, the power supply 26 may also be implemented through the use of a capacitor. It may be possible to have the power supply 26 be capable of being charged or re-charged through the use of an external power source, such as a wired and/or wireless battery charger or charging field.

According to an embodiment, the communications module 18 facilitates wired or wireless communication between the wearable device 120 and other devices and/or networks, such as network 140. The communication module 18 may be facilitated through the use of one or more antennas. The communication module 18 can facilitate communication with one or more networks or with other devices, for example, by using wireless methods that are known, including but not limited to Wi-Fi, Bluetooth, 3G, 4G, LTE, and/or ZigBee, among others.

The wearable device also comprises one or more integrated sensors 14. The one or more sensors 14 are used to monitor and obtain sensor data, and thus evaluate a condition of the user and/or a parameter of the environment in which the user is located. The one or more sensors 14 may comprise, for example, a sensor configured to measure, determine, or derive blood pressure, heart rate, heart rhythm, heart rate variability, pulse, blood oxygen, body temperature, skin temperature, acceleration, body posture, vitamin levels, respiratory rate, heart sound, breathing sound, movement speed, movement acceleration, muscle tension, brain activity, sleep stages, sleep onset latency, sleep duration, steps walked or ran, skin moisture, sweat detection, sweat composition, nerve firings, or similar health measurements, and other sensors known in the art. Accordingly, the one or more sensors 14 may be a sphygmomanometer, photoplethysmogram (PPG) sensor, ECG-based heart rate monitor, pulse oximeter, thermometer, electromyography sensor, electroencephalography sensors, accelerometer, microphone, pedometer, electromagnetic sensor, camera, pressure sensor, and/or any other type of sensor.

The wearable device may comprise a user interface 16 to receive input from the consumer 110. The user interface may be a button or multiple buttons, a microphone, a key stroke input, or any of a variety of other inputs. According to another embodiment, consumer 110 may provide the input remotely via a computer which communicates the input to the wearable device via network 140.

According to an embodiment, one or more combinations of sensors might be used to derive a physiological parameter. For example, a PPG sensor on the finger or wrist can be used in combination with an ECG-based patch on the chest in order to derive pulse arrival time, which can be utilized as a surrogate measure for blood pressure. Additionally, a single sensor can be used to derive various physiological parameters. For example, an SpO₂ sensor might be used to measure oxygen saturation and measure heart rate. An ECG-based patch on the chest could be used to measure heart rhythm and measure the width of the P-wave, height of the R peak, and/or other ECG characteristics. A normal or thermal camera can be used to measure one or more of activity, pulse rate, blood oxygenation, respiration rate, and skin temperature. Another method to measure activity and vital signs like heart rate and respiration rate is through radar. These parameters can also be measured with pressure sensors in the bed or on the seat of a chair or toilet. According to yet another embodiment, blood pressure could be measured via an oscillometric method with an arm cuff, or, less obtrusively, estimated from a surrogate measurement such as the above-mentioned pulse arrival time. As used herein, the term “physiological sensor data” will be understood to encompass such derived physiological data, as well as the raw sensor data.

The wearable device 120 also comprises a display 20 which is utilized by the wearable device to provide information or otherwise facilitate interaction between the user and the wearable device. The display may be, for example, an LED-based, LCD-based, or e-paper type display. In other embodiments, the display may be a touch screen display that allows the user to directly interact with the wearable device through physical contact and/or gestures.

The components of the wearable device 120 in FIG. 2 can be connected via a single bus, and/or through one or more data transport means. As such, some components may be connected via a local microprocessor bus, and others may be connected via one or more input/output (I/O) buses.

The coffee consumption monitoring device 150 in FIG. 2 can be, for example, any device that monitors or provides information about the consumer's consumption of coffee or the beverage of interest. For example, coffee consumption monitoring device 150 may be a coffee maker. The coffee maker can track coffee made and/or consumed by the consumer. For example, the coffee maker may comprise a volume sensor, a pour sensor, a motion sensor, or any of a variety of other monitors or sensors to obtain information about coffee creation and/or consumption. The coffee consumption monitoring device 150 may therefore comprise a communications module to communicate the information via network 140 to wearable device 120 and/or another computer or server 160.

According to another embodiment, coffee consumption monitoring device 150 may be an intelligent handheld device associated with coffee consumption, such as a coffee cup, mug, or thermos. The coffee cup could comprise, for example, temperature, motion, and/or other sensors to obtain information about coffee consumption by the consumer. The coffee cup may also comprise a communications module to communicate the information via network 140 to wearable device 120 and/or another computer or server 160.

According to an embodiment of system 200, computer or server 160 is a data repository and/or analysis engine. For example, as described or otherwise envisioned herein, the computer or server 160 may receive and analyze coffee consumption and/or physiological information in order to create personalized consumption recommendations. According to another embodiment, however, the functionality of computer or server 160 may be performed entirely by wearable device 120.

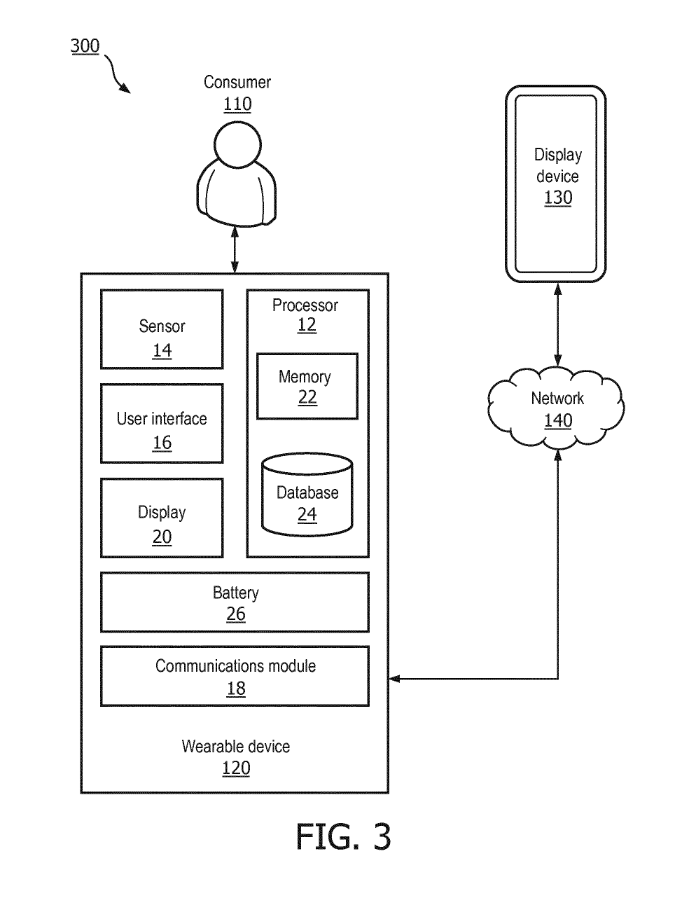

Referring to FIG. 3, in one embodiment, is a 300 comprising a consumer 110 and a wearable device 120. The consumer 110 is any individual that has or is planning to consume a beverage, food, and/or medicine that may affect the user physiologically. The wearable device 120 in system 200 can be any of the devices described or otherwise envisioned herein. For example, wearable device 120 may comprise one or more of a processor 12, sensor 14, user interface 16, communications module 18, display 20, memory 22, database 24, and/or battery or power supply 26. The wearable device 120 is configured or programmed to obtain physiological data about the consumer 110.

According to an embodiment, the wearable device 120 may monitor or provide information about the consumer's consumption of coffee or the beverage of interest. For example, the wearable device 120 may recognize repeated hand movements associated with prolonged or periodic sipping or drinking of the beverage of interest.

The wearable device may comprise a user interface 16 to receive input from the consumer 110, such as input about one or more personal goals. The user interface may be a button or multiple buttons, a microphone, a key stroke input, or any of a variety of other inputs. According to another embodiment, consumer 110 may provide the input remotely via a computer which communicates the input to the wearable device via network 140.

The wearable device 120 can also comprise a display 20 which is utilized by the wearable device to provide information or otherwise facilitate interaction between the user and the wearable device. The display may be, for example, an LED-based, LCD-based, or e-paper type display. In other embodiments, the display may be a touch screen display that allows the user to directly interact with the wearable device through physical contact and/or gestures.

According to another embodiment, however, system 300 utilizes display device 130 in whole or in part to receive and/or convey information to and from the consumer. The device 130 may be, for example, a smartphone or other portable device. According to yet another embodiment, the device 130 may be a computer such as a desktop, laptop, tablet, or other permanent or semi-permanent computing device. The device 130 may receive input from the consumer and may also display information to the consumer, as described or otherwise envisioned herein.

According to an embodiment, one or more of the maximum and/or minimum thresholds can be modified based on an event that occurs or is schedule or predicted to occur, or a physiological event or parameter detected. For example, the threshold may be modified when a new entry is entered into the user's calendar. The entry is detected by the system, and the information is utilized to modify one or more of the maximum and/or minimum thresholds. For example, the entry of a late-night meeting may impact the maximum and/or minimum caffeine threshold in order to provide caffeination later into the evening. Similarly, an early-morning meeting may result in an increase of the minimum and/or maximum caffeine threshold in order to provide caffeination earlier in the morning. According to another embodiment, the detection of an arrhythmia, such as by a wearable or other monitor or sensor, may be utilized to modify one or more of the maximum and/or minimum thresholds. For example, the detected arrhythmia may significantly lower the maximum caffeine threshold in order to prevent over-caffeination.

According to another embodiment, one or more of the maximum and/or minimum thresholds can be modified based on a prioritization of the user's goals. Accordingly, the system will consider the goals in light of physiological and/or consumption information about the user, and will prioritize the goals in order to generate a maximum and/or minimum threshold. In some embodiments, two or more goals may result in identical or similar thresholds, while in other embodiments two or more goals may result in different thresholds. When the user's goals result in different thresholds, the user and/or the system must prioritize those thresholds in order to select one or a plurality of different possible thresholds. For example, the user may designate goals such as “improve sleep quality” and “increase wakefulness in the evenings.” These goals may result in conflicting thresholds, where improving sleep quality decreases maximum and/or minimum thresholds later in the day, and increasing evening wakefulness may increase maximum and/or minimum thresholds later in the day. The user may designate a goal priority, or the system may determine a goal prioritization based on pre-programmed priority lists or decision-making processes, and/or on prior or current physiological data.

As another example, the user may designate goals such as “improve mid-afternoon caffeination” in order to stay awake, while also designate the goal of “prevent migraines.” These goals may result in conflicting thresholds, especially if caffeine can trigger or worsen the user's migraine. Accordingly, the system may determine (alone or based on user information) that preventing migraines takes priority over improving mid-afternoon caffeine levels. In the morning or early afternoon, the system may detect that a migraine is likely to happen in the next hour. This could be based on direct user input, and/or could be based on the user's physiological data. For example, historical physiological data may identify indicators that present just prior to a migraine. Once the possible migraine is detected, the system will modify the maximum and/or minimum thresholds in order to lower overall caffeine levels in an attempt to prevent the migraine, even though this directly conflicts with the goal of improving mid-afternoon caffeine levels. Once the threat of a migraine is over, the system can again modify the maximum and/or minimum thresholds in order to increase overall caffeine levels in an attempt to improve mid-afternoon caffeine levels.

In another embodiment, the system makes a compromise for a threshold when different goals conflict. As one example, one of the user's goals is to lose weight and another to have the best possible sleep. During the day, if the system detects that the user hasn't eaten for more than three hours, it advices to drink at least one cup of coffee in order to avoid binge eating. During the evening, when the user had a normal dinner and there is thus no reason to predict binge eating, the system advices to not drink coffee anymore in order to have the best possible sleep. However, in case the user hasn't eaten for more than three hours in the evening, the minimum threshold for the goal to lose weight (namely one cup of coffee to avoid binge eating) conflicts with the maximum threshold to get the best possible sleep (namely no coffee). The system could then make a compromise and advise to drink half a cup of coffee. It could even make a weighted compromise; for example, if the goal of having the best possible sleep has a higher priority than the goal to lose weight, it could advice to take only two sips of coffee.

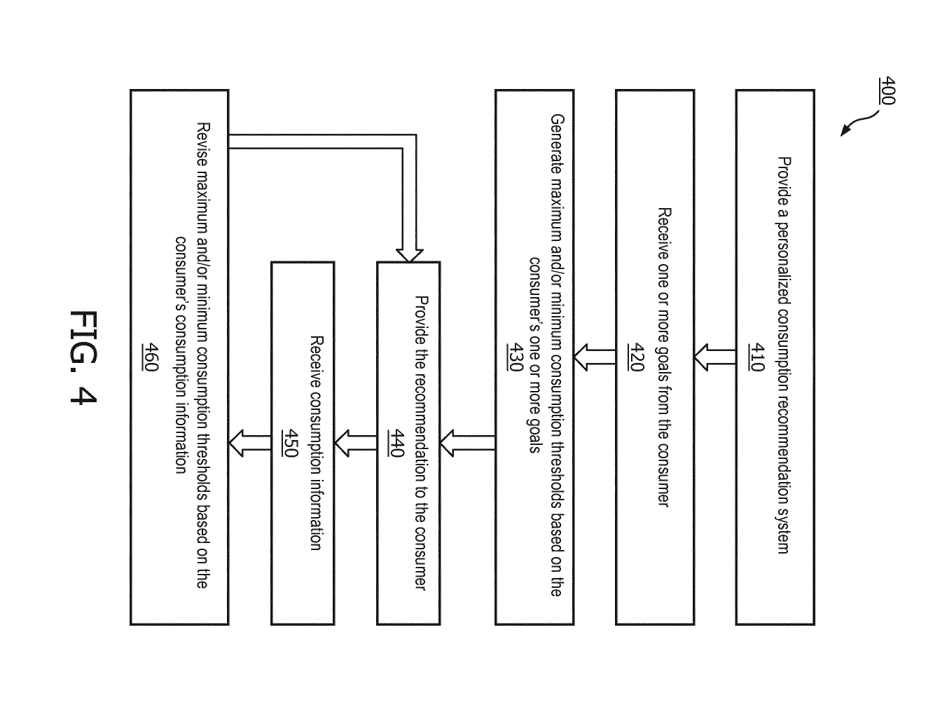

Referring to FIG. 4, in one embodiment, is a method 400 for providing individualized thresholds for food, beverage, and/or medicine consumption based on consumption information and goals about the consumer. At step 410 of the method, a personalized consumption recommendation system or device is provided. The recommendation system may be any of the systems described or otherwise envisioned herein, including but not limited to system 100 in FIG. 1, system 200 in FIG. 2, and/or system 300 in FIG. 3.

At step 420 of the method, the personalized consumption recommendation system or device receives one or more goals from or about the consumer. The goals may be short-term and/or long-term goals. For example, the goal may be to lower blood pressure, avoid migraines or headaches, avoid arrhythmias, increase activity levels, stay active, improve sleep, avoid, lessen, or cure coffee addiction, and/or lose weight, among many, many other possible goals. As yet additional example, the goal could be to reduce sleep onset latency, stay awake until a certain time, or live as healthy as possible in general.

The goal can be entered into or provided to the system via a variety of mechanisms. For example, the consumer can choose from among a variety of predetermined goals during registration and/or during use of the system or method. A list of goals may be provided and selected from by means of a user input or interface.

At step 430 of the method, the personalized consumption recommendation system or device generates one or more beverage, food, or medicine consumption thresholds based on the consumer's one or more goals. For example, the system may generate a maximum and/or minimum beverage, food, or medicine consumption threshold. The minimum threshold might be, for example, a level of coffee consumption and/or caffeination above which the consumer should or prefers to be. The maximum threshold might be, for example, a level of coffee consumption and/or caffeination below which the consumer should be to achieve the one or more goals set by the consumer.

For many consumers, drinking coffee late at night is undesirable because it will lead to increased sleep onset latency. However, other consumers may need coffee late at night because lack of coffee will wake them up later in the night because their body is craving coffee. The one or more consumption thresholds may be based on, for example, the relationship between the timing and/or dose of coffee intake in the evening and sleep quality at night, and will provide thresholds for coffee intake in such a way as to optimize sleep quality. For many consumers, drinking coffee in high amounts may cause stress, while other consumers might experience stress as a result of coffee deprivation. Accordingly, the system could consider the consumer's individualized response to coffee and coffee deprivation. For example, the system can analyze the balance between coffee intake and stress levels, and can gradually—such as day-by-day or week-by-week—coach the user towards more optimized coffee intake to reach the user's goals.

According to an embodiment, a machine learning algorithm (e.g., gradient descent) may be used to train a predictive model (e.g. a regression model or a neural network) for each such consumer goal for use in determining the effect of a given caffeine level at a given time on the overall success of the goal. Accordingly, a single value for each threshold may be utilized, even though each of two or more goals may be individually associated with its own set of thresholds. For example, a maximum threshold at 9 AM for falling asleep at bedtime may be at a high level, while the maximum threshold for avoiding migraines may be at a mid-level; the mid-level threshold would then be chosen as the overall maximum threshold.

According to yet another embodiment, the personalized consumption recommendation system or device also utilizes other information when generating the recommendation. For example, the system can consider the consumer's consumption preferences, such as the desired coffee times and strength, the user's health issues or circumstances, such as pregnancy and weakness of stomach, and/or a variety of other parameters or information. According to an embodiment, the one or more beverage, food, or medicine consumption thresholds may also be based in whole or in part on physiological information about the consumer. For a system comprising a wearable device 120, the physiological information can be generated, collected, and/or stored by the one or more sensors as described herein. This physiological information will often be generated and collected for many different uses, one of which might be the consumption recommendation method. According to an embodiment, instead of only one wearable device utilized to capture physiological signals and/or activity data, a combination of wearables or remote devices can be used, such as a watch, ear plugs, a patch on the chest, and a camera, among many other possible devices and sensors.

According to another embodiment, the physiological information can be generated and collected by a device other than a wearable device. For example, the physiological information can be generated and collected by a coffee maker, an intelligent beverage cup, or other device with a sensor. The device will comprise one or more sensors 14 configured to obtain physiological information about the consumer. For example, the device may comprise an electrode to obtain heart rate, a camera to obtain one or more vital signs such as blood pressure, pulse, and breathing rate, and/or any of a variety of other sensors. For a smart coffee maker, for example, there may be a sensor or camera on the handle or a button of the device. For a smart coffee cup, for example, there may be a sensor on the handle of the cup. Many other devices and configurations are possible.

The physiological data may be collected continuously, may be collected only during a time period when the beverage is being consumed and processed by the body, or may be collected continuously but only stored or analyzed during time periods when the beverage is being consumed and processed by the body. According to an embodiment, when monitoring is only done during certain time periods, it is can be done from just before drinking coffee, in order to obtain a baseline, until a predetermined time period after finishing the coffee, to see the response. Monitoring could for example be triggered to start by a button press on the coffee machine, which also starts the brewing of coffee. Alternatively, the measurement can be done at a fixed time each day, such as every morning before the first cup of coffee or only at night, in order to observe a general trend in health changes associated with altered coffee intake.

According to an embodiment, the system or device may also be configured to receive individualized feedback from the consumer about his or her physiology, psychology, well-being, or other feedback. For example, the smartphone or wearable device might be configured to obtain or receive custom input from or about the consumer regarding the quality of sleep, feeling of stress, feeling of well-being, having stomach pain, having a migraine, being pregnant, having an injury, weakness of stomach, a morbidity such as kidney or liver function, and/or any of a wide variety of other inputs. Accordingly, the system may factor a physical and/or mental status into the recommendation.

At step 440 of the method, the personalized consumption recommendation system or device pushes the one or more beverage, food, or medicine consumption thresholds to the consumer. For example, the thresholds can be provided to the consumer via a wearable device, via the coffee machine, on a local computer monitor or TV screen, or on another device such as a smartphone or tablet. As another embodiment, the wearable device, smartphone, or other display device provides a countdown until the consumer should consume additional coffee. For example, the wearable device, smartphone, or other display device can depict a countdown of two hours until additional coffee is recommended, after the consumer has consumed two cups of coffee within the past hour.

At step 450 of the method, food, beverage, and/or medicine consumption information is generated and received by the personalized consumption recommendation system or device. In the case of coffee, for example, the coffee consumption information can include information about when the coffee was consumed (including the start and stop times for the consumption), how much coffee was consumed, what was or was not added to the coffee, and/or the strength of the coffee. According to an embodiment, the consumption information can be generated or shared by many different devices, and can be obtained by an analysis processor via a network such as a wired connection, WiFi, Bluetooth, NFC, or any other wired or wireless connection. Alternatively, the consumption information can be sent to the cloud, where it can be stored and retrieved as needed.

The consumer can provide consumption information directly via a user interface. For example, the user can enter information about the amount, strength, and timing of coffee intake on a computing device such as the consumer's wearable device 120, a smartphone, tablet, PC, and/or any other computing device. The data can be entered at home, at work, in a coffee shop, on the go, or anywhere. In a scenario where a home coffee maker is connected to the system and automatically collects information, while coffee consumption outside the home must be recorded, the consumer will only enter information for coffee consumed when outside the home. The information may be entered by the consumer via selection of menus, automated selections, buttons, voice recognition, typing, or other user interface mechanisms.

The coffee machine itself may provide coffee consumption information. According to an embodiment, every time a coffee machine brews coffee, this can be used as time stamp for coffee intake after which a physiological response of the consumer can be measured. The coffee machine at home is connected to the cloud or directly to a wearable device or other computing device, and collects the information needed for personalized recommendations. Preferably, the consumer's coffee machine at work is similarly connected to the cloud or directly to the consumer's wearable device or other computing device. Alternatively, other mechanisms as described or otherwise envisioned herein will be utilized in settings or locations where a coffee machine is not collecting and sharing consumption information.

If the consumer is the only person who utilizes the coffee machine, or the only person that consumes the coffee, then the machine may automatically record all information for that consumer. However, the coffee machine may also be utilized by a spouse, family member, roommate, co-worker, or other individual within the home or office. This scenario requires extra measures to enable registration of all coffee intake and exclude intake by others. For example, in order to discriminate the desired consumer from other people using the same coffee machine, the consumer can enter his or her personal code on the coffee machine when starting the brewing of the coffee, or when pouring a cup or portion of coffee. Alternatively, the coffee machine might automatically recognize the consumer, for example by finger print recognition on the buttons, by face recognition with a camera, or by proximity detection of the user's wearable, which might, for example, contain an RFID tag or other recognition means. Recognition of the consumer by the coffee machine, either due to manual identification or automated identification, could, in addition to being used to obtain information on coffee intake, also be used to automatically brew the coffee blend, amount, and/or strength of the consumer's preference, or, as described herein, to automatically brew the blend, amount, and/or strength that is being recommend to the consumer by the personalized recommendation advisor.

According to another embodiment, the wearable device 120 may provide the consumption information. For example, a hand- or wrist-worn wearable such as a ring or watch with a motion sensor like an accelerometer or gyroscope can recognize the arm and/or hand movement of drinking, and the system can recognize not only the time of consumption, but possibly even the amount based on the number of movements, and/or on another quantitative or qualitative measurement of movement. This information might also be combined with a known response to drinking coffee such as an increase in heart rate, especially when the system has already learned how the user would normally respond to drinking coffee. This might differentiate between the consumer drinking coffee versus drinking another beverage such as water. A combination of physiological signs and/or motion recognition of drinking coffee could then lead to automatic recognition of when the user is drinking coffee.

According to another embodiment, the system may include an intelligent handheld device associated with coffee consumption, such as a coffee cup, mug, or thermos. The coffee cup could comprise, for example, temperature, motion, and/or other sensors to obtain information about coffee consumption by the consumer. The cup may comprise an RFID tag, which is recognized by the coffee machine and/or the wearable device. Accordingly, the cup may not be limited to use by one consumer, but may only be limited to the consumer at the time of drinking Thus, the user's spouse, coworker, roommate, or other consumer could use the cup. The coffee cup could also contain a sensor that detects coffee in the cup, for example by a thermometer, optionally combined with an optical sensor to discriminate between coffee and tea. The intelligent cup might even measure how fast the beverage is being consumed. A gyroscope or accelerometer in the cup might be used to detect the motion of bringing the cup to the mouth and drinking from it. In addition, the cup might also contain sensors to measure physiological signals while the user holds his cup, such as a photoplethysmogram (PPG) sensor in the handgrip, among many other types of possible sensors.

At step 460 of the method, the personalized consumption recommendation system or device analyzes the consumer's consumption information and updates the one or more beverage, food, or medicine consumption thresholds. For example, the one or more consumption thresholds can be updated several times throughout a 24-hour period. As another example, the threshold(s) may be updated at set time periods, continually, or in response to certain predetermined events, such as when the beverage, food, or medicine being tracked is consumed, when there is a sharp physiological or other environmental change, or in response to a variety of other triggers. The method then returns to step 440 of the method, and the personalized consumption recommendation system or device pushes the one or more consumption thresholds to the consumer.

Instead of displaying the thresholds or recommendation on the wearable device, smartphone, tablet, computer, or TV screen, for example, environmental lighting could also be used to create an atmosphere that either promotes or discourages drinking coffee. Many other mechanisms for controlling, encouraging, or discouraging consumption are possible. For example, the system may override the coffee maker to only work based on the recommendations of the system.

According to an embodiment, the personalized consumption recommendation system or device optically collects information about the amount, strength, and/or timing of coffee consumed per day in order to generate and/or share information such as totals, averages, and trends to the consumer, a coach, a physician, a cardiologist, or another medical specialist to provide insight in habits and change in habits. For example, the shared information can reveal periods when the consumer is demanding too much from his or her body, resulting in tiredness, which the consumer compensates for by consuming more coffee, and/or to coach the consumer for further lifestyle improvement to reach the consumer's goals.

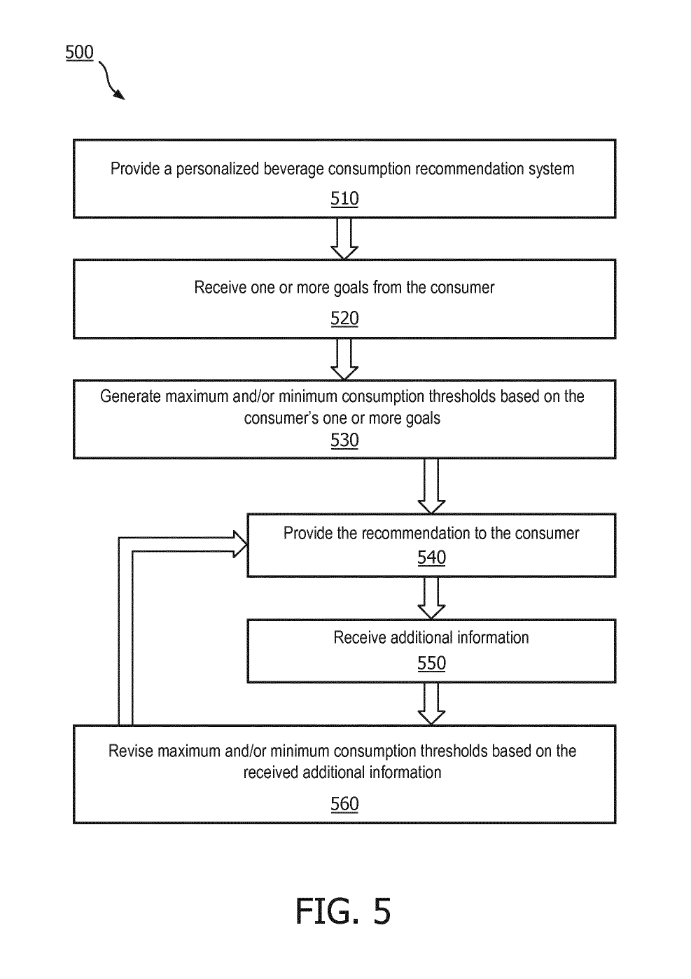

Referring to FIG. 5, in one embodiment, is a flowchart of a method 500 for providing one or more individualized thresholds for food, beverage, and/or medicine consumption based on consumption information and goals about the consumer. At step 510 of the method, a personalized consumption recommendation system or device is provided. The recommendation system may be any of the systems described or otherwise envisioned herein, including but not limited to system 100 in FIG. 1, system 200 in FIG. 2, and/or system 300 in FIG. 3.

At step 520 of the method, the personalized consumption recommendation system or device receives one or more goals from or about the consumer. The goals may be short-term and/or long-term goals. For example, the goal may be to lower blood pressure, avoid migraines or headaches, avoid arrhythmias, increase activity levels, stay active, improve sleep, avoid, lessen, or cure coffee addiction, and/or lose weight, among many, many other possible goals. As yet additional example, the goal could be to reduce sleep onset latency, stay awake until a certain time, or live as healthy as possible in general.

At step 530 of the method, the personalized consumption recommendation system or device generates one or more beverage, food, or medicine consumption thresholds based on the consumer's one or more goals. For example, the system may generate a maximum and/or minimum beverage, food, or medicine consumption threshold. The minimum threshold might be, for example, a level of coffee consumption and/or caffeination above which the consumer should or prefers to be. The maximum threshold might be, for example, a level of coffee consumption and/or caffeination below which the consumer should be to achieve the one or more goals set by the consumer.

At step 540 of the method, the personalized consumption recommendation system or device pushes the one or more beverage, food, or medicine consumption thresholds to the consumer. For example, the thresholds can be provided to the consumer via a wearable device, via the coffee machine, on a local computer monitor or TV screen, or on another device such as a smartphone or tablet.

At step 550 of the method, additional information is received by the personalized consumption recommendation system or device. This additional information can take many different forms, but is generally any information that has the potential to impact the recommended one or more thresholds. For example, the additional information received by the personalized consumption recommendation system or device could be a change in the user's calendar or agenda, information about a detected physiological parameter or health event such as a migraine or an increased or decreased activity level, and other information.

At step 560 of the method, the personalized consumption recommendation system or device analyzes the consumer's additional information and updates the one or more beverage, food, or medicine consumption thresholds. According to an embodiment, one or more of the maximum and/or minimum thresholds can be modified based on an event that occurs or is schedule or predicted to occur, or a physiological event or parameter detected. For example, the threshold may be modified when a new entry is entered into the user's calendar. The entry is detected by the system, and the information is utilized to modify one or more of the maximum and/or minimum thresholds. For example, the entry of a late-night meeting may impact the maximum and/or minimum caffeine threshold in order to provide caffeination later into the evening. Similarly, an early-morning meeting may result in an increase of the minimum and/or maximum caffeine threshold in order to provide caffeination earlier in the morning. According to another embodiment, the detection of an arrhythmia, such as by a wearable or other monitor or sensor, may be utilized to modify one or more of the maximum and/or minimum thresholds. For example, the detected arrhythmia may significantly lower the maximum caffeine threshold in order to prevent over-caffeination

The method then returns to step 540 of the method, and the personalized consumption recommendation system or device can push the one or more consumption thresholds to the consumer.

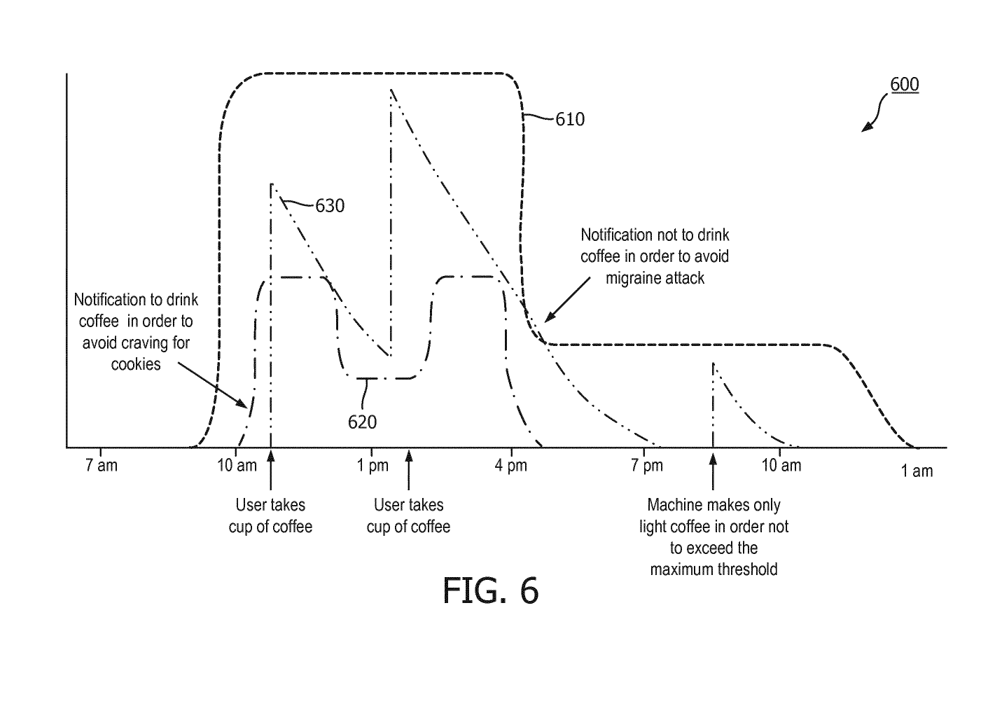

Referring to FIG. 6, in one embodiment, is a 24-hour timeline 600 with beverage consumption and thresholds. According to this embodiment, the consumer is overweight and prone to migraines, and therefore established a list of goals: (1) avoid migraines; (2) lose weight; (3) sleep well; and (4) avoid coffee addiction. Many other goals are possible, but these are selected for the embodiment depicted in FIG. 6. As shown in FIG. 6, the maximum threshold 610 and minimum threshold 620 vary throughout the course of the day depending on at least the consumer's goals and consumption. The amount or concentration of caffeine in the body 630 is also shown.

On this particular 24-hour period depicted in FIG. 6, the consumer wakes up at 7 AM. To avoid becoming addicted to coffee, drinking coffee in the first two hours after waking up should be avoided. Accordingly, the system generates a maximum and minimum threshold of “0” until 9 AM, which increases after 9 AM, signaling to the consumer that coffee can be consumed. A maximum or minimum threshold of “0” may or may not be shown to the consumer. For example, according to an embodiment, the consumer may be aware that a maximum or minimum threshold is “0” whenever it isn't displayed or provided.

The system may be programmed or may learn to know that if the consumer does not consume any caffeine by 11 AM, the consumer will be craving cookies or snacks and is more likely to consume food that the consumer shouldn't consume, particularly in light of the goal of losing weight. The system may know, therefore, that if the consumer drinks coffee between 10 and 11 AM, the consumer will not crave food until lunch. As a result, the minimum threshold 620 increases at 10 AM. Due to the increase of the minimum threshold 620 at 10 AM, the amount of caffeine in the body 630 is then lower than the minimum threshold, and the consumer is notified that coffee should be consumed. The consumer consumes a cup of coffee, thereby receiving an amount of caffeine in the body between the minimum and maximum thresholds. After that, the amount of caffeine in the body slowly reduces as the caffeine is metabolized by the consumer's body.

Around lunch time, the minimum threshold is decreased and the consumer eats lunch. At 1:30 PM, the consumer consumes another cup of coffee. Accordingly, the amount of caffeine in the body is now the sum of the caffeine that was still left in the body from the previous cup of coffee, plus the caffeine from the new cup of coffee. The amount of caffeine in the body 630 is still appropriate, however, as the level is still between the minimum 620 and maximum 610 thresholds.

At 2:30 PM, the minimum threshold 620 is again increased by the system to avoid craving for cookies around 3 PM. No notification is given at this time, however, because even with the increased minimum threshold the amount of coffee in the body is still above the minimum threshold.

At 4 PM, the system detects that the consumer is close to getting a migraine. To avoid the migraine, the system knows that the consumer should refrain from drinking coffee. Accordingly, the system reduces the maximum threshold 610 significantly, and the consumer receives a notification that the current caffeine level is higher than the maximum threshold. The consumer doesn't drink coffee, and as the body metabolizes the caffeine, the level of caffeine soon settles back between the minimum and maximum thresholds. After the migraine threat has disappeared, the maximum threshold stays relatively low in order to give the consumer a good night's sleep.

At 7:30 PM, however, the consumer decides to drink coffee. The coffee machine, which is part of the system, brews the consumer only a light cup of coffee or a decaffeinated cup of coffee, because a normal cup of coffee would place the consumer above the maximum threshold, which would lead to a high sleep onset latency.

Instead of the amount of caffeine in the body 630 (which jumps immediately up after coffee intake and declines after that), the system could show (or work internally with) a measure for the response of the body to caffeine. Such a measure could be derived from the changed heart rate, changed blood pressure, or other variations due to coffee intake or combinations of those. As it usually takes some time before the effect of caffeine kicks in, such a measure will increase after coffee intake, peak after some time, for example 20 minutes, and then decline slowly. In case a measure for the response of the body is used instead of the amount of caffeine in the body 630, the threshold 610, 620 will accordingly be based on the measure for the response of the body.

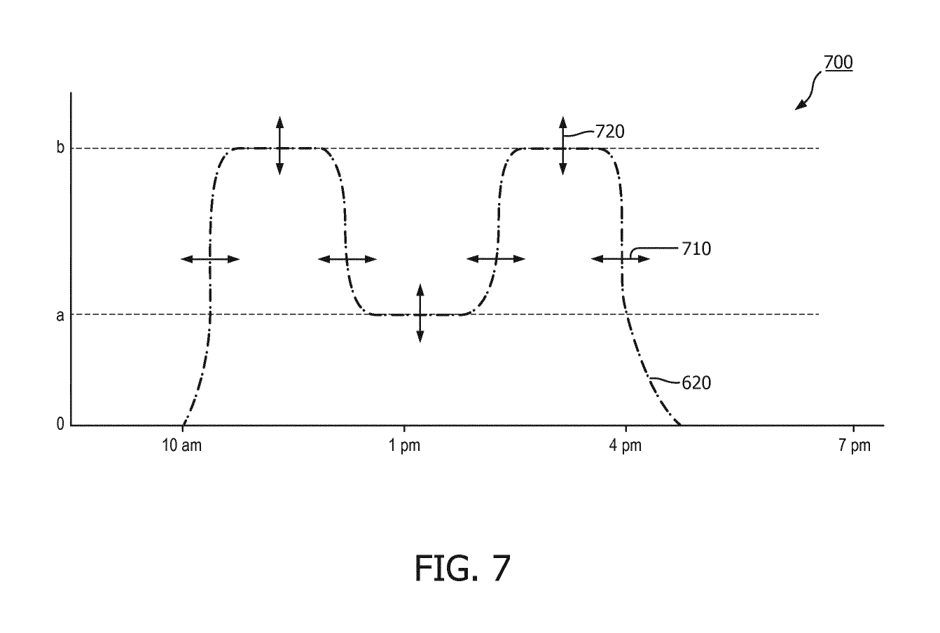

Referring to FIG. 7, in one embodiment, is an example of a just the minimum threshold 620 throughout most of a 24-hour period. The arrows in FIG. 7, such as arrows 710 and 720, indicate shifts in the minimum threshold 620 that are possible for various reasons as described herein. In this example, the minimum threshold 620 is being analyzed and updated throughout the day, and it is moving between three levels: (1) level “0”, such as prior to 10 AM, which may be desirable at night close to bedtime, during the night, in the morning when there is not yet a reason to drink coffee, and when the person is close to getting migraine, among many other possible times; (2) level “a,” which is the minimum amount, level, or concentration of caffeine needed to stay alert and receive the beneficial effects of coffee; and (3) level “b,” which is the minimum amount, level, or concentration of caffeine required to suppress craving for cookies. According to yet another embodiment, the minimum threshold 620 could be updated throughout a 24-hour period to be at four, five, or any number of different levels or amounts.

According to an embodiment, the level of the minimum threshold 620 can vary slightly in the long term, such as the changes depicted by arrows 720, by learning the physiological caffeine response of the consumer and/or by getting renewed insight from literature or from data produced by other consumers of the system, algorithm, or program. A shorter-term variation might be introduced by looking at one or more current physiological parameters of the consumer. As an example, if the consumer's blood pressure in the morning turns out to be much lower than normal, the consumer might be allowed or required to drink more coffee. The estimate for blood pressure or any other metric can be measured continuously or could be measured periodically. As another example, if the system is being utilized to provide thresholds and recommendations for salt intake, and the consumer has a low blood pressure measurement at some point, the maximum and/or minimum threshold may be increased by the system.

According to another embodiment, the timing of the change to the minimum threshold 620 can vary slightly in the long term, such as the changes depicted by arrows 710, due to a variety of factors. For example, the system could learn the physiological response of the consumer or renewed insight could be obtained from literature or from data produced by other consumers of the system. As another example, the system might respond to a health-related event, such as an upcoming migraine attack, which is detected from the measured physiological signals of the consumer or by consumer input. As another example, the system might incorporate a scheduled event, such as a meeting or a running race, for which the consumer needs to be alert, and for which the system gets the information, such as via the consumer's calendar. As another example, the system might review at the current physiological parameters; as just one example, if the blood pressure is low, the consumer might be allowed or required to drink coffee earlier than normal. As another example, the system might take into account the amount of caffeine present in the body already. This is especially applicable to the maximum threshold; if the level of caffeine in the body is already high and the maximum threshold is going to be lowered to a substantially lower level, a more gradual lowering could be followed instead of a jump, so that the amount of caffeine in the body will not get higher than the upper threshold. This could also apply to the minimum threshold; when the minimum was at level zero and would increase to a much higher level while the caffeine in the consumer's body was zero instead of already somewhere between zero and the maximum threshold, the minimum threshold could, instead of jumping directly to the high level, follow a two-step approach with a level in between, so that the consumer can drink two cups of coffee with some time in between instead of having to drink both cups at once.

In various embodiments, the consumer may be provided with a simple interface for conveying their current caffeination levels versus the personalized thresholds. For example, the chart 600 of FIG. 6 (or a chart similar thereto) may be displayed to the user and gradually completed as the day progresses, thereby enabling the use to quickly ascertain their current caffeination level versus their current thresholds, as well as historic values for these parameters (e.g. historic values for the current day, the previous week, etc. depending on the length of the x-axis). In an alternative embodiment, the display may only convey a snapshot of the current time. For example, a single bar, volume, or other metaphor to convey amount of caffeine in the consumer's body may be displayed along with lines, boundaries, or other indications of the current thresholds. In some such embodiments, a timeline may be useful to shuttle to previous points in time and view the snapshot at such earlier times. In some embodiments, the visualization may also show a projection of future states. For example, in the chart embodiment of FIG. 6, the future thresholds may be plotted past the current time. In some such embodiments, a prediction of the future caffeine levels may also be plotted past the current time. In one or more of the snapshot embodiments, a timeline may be provided to shuttle not only to previous points in time but also future points in time to show a projected snapshot at that time. In some embodiments, the projected states may simply show the future caffeine state if the consumer does not drink any more coffee between the current point and the projected point, while in other embodiments the future state may take into account one or more additional cups of coffee based on previously noted consumer habits, explicit indication by the consumer of plans to drink coffee, or simply as one of multiple simultaneously displayed or selectable alternatives (no coffee, one cup of coffee, and two cups of coffee). To enable such functionality, the system may include at least one model for predicting future caffeinated states. For example, a model may be trained based on population data, data gathered from the specific user, or combinations thereof to determine how quickly caffeine is metabolized and removed from the consumer's system (thereby facilitating prediction of the downward slopes in FIG. 6). In a similar manner, a model may be trained to predict the effect of a cup of coffee of the caffeine line 630 in view of a reported coffee consumption event or to predict, based on user habits, likely future coffee consumptions.

Accordingly, as described herein, variations in the height of a threshold, indicated by changes to arrows 720, and the timing of a change from one threshold level to another, indicated by changes to arrows 710, can be based on the consumer's goals, the consumer's consumption of the tracked item, and/or on the consumer's physiological state. The changes might also be based in whole or in part on learning the consumer's response to caffeine. For example, the levels and timing might, during the learning process in the first couple of weeks that the consumer uses the system, be based on a population rather than on the individual consumer. As inadequate advice, especially in these first weeks of use, could misdirect the consumer and prevent the consumer from relying on the system, the system could be designed such that during the first weeks it will only learn and will not give any advice.

According to an embodiment, the recommendation system or device optionally collects information about the amount, strength, and/or timing of the item consumed per day in order to generate and/or share information such as totals, averages, and trends to the consumer, a coach, a physician, a cardiologist, or another medical specialist to provide insight in habits and change in habits. For example, the shared information can reveal periods when the consumer is demanding too much from his or her body, resulting in tiredness, which the consumer compensates for by consuming more coffee, and/or to coach the user for further lifestyle improvement to reach the consumer's goals.

As one example, the recommendation system or device can collect physiological data and/or consumption data for a period of time about a particular user and can store it and/or send it to the user's physician, trainer, physical therapist, dietician, or other specialist for analysis. A cardiologist, for example, may require that a patient utilize the recommendation system for a period of time in order to track the user's consumption of an item such as coffee and monitor the user's response with the physiological data or with other physiological data such as a heart monitor. The cardiologist could then analyze the data and provide information resulting in adjustment of the recommendations made by the system. If, for example, the cardiologist recommends less caffeine consumption, the recommendation system or device will consider that information when making recommendations to the user. Similarly, a personal trainer may receive physiological data and consumption data about an item in order to track the efficiency or outcome of a user's workout regimen.

Although the personalized consumption recommendation method, systems, and device is described primarily in conjunction with coffee consumption, it should be recognized that any consumed beverage or food, and some medications, treatments, or remedies, could be utilized in this system. For example, the beverage could be tea, alcohol, soda, or any of a wide variety of other beverages. The food could be anything consumed by the consumer, and will typically be something that the consumer has a desire to track or control, such as sugar consumption or salt intake, among many others. Advice on salt intake could be important for people with hypertension, heart failure, or kidney failure, whereas advice on water intake could for example be important for elderly to prevent dehydration. The medications, treatments, or remedies could be aspirin, a pain reliever, antibiotic, salve, cream, lotion, or any of a variety of others.

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of” “only one of,” or “exactly one of.” “Consisting essentially of” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

1. A computer-implemented method for providing a consumption threshold to a user about consumption of an item, the method comprising the steps of:
receiving one or more goals from the user;
generating, based on the received one or more goals from the user, a consumption threshold;
providing the generated consumption threshold to the user;
receiving information about the user;
updating, based on the information about the user, the consumption threshold; and
providing the updated consumption threshold to the user.

2. The method of claim 1, wherein the information about the user comprises at least one of the user's consumption of the item, physiological data about the user, and the user's schedule.

3. The method of claim 1, wherein the consumption threshold is updated a plurality of times during a 24-hour period.

4. The method of claim 1, wherein the consumption threshold comprises a minimum consumption threshold, a maximum consumption threshold, and/or an optimal consumption threshold.

5. The method of claim 1, wherein the information about the user comprises the amount of the item consumed and the time of consumption.

6. The method of claim 1, wherein the updating step is based at least in part on either an activity level of the item by the user or an estimated amount of the item in the user's body.

7. (canceled)

8. The method of claim 1, further comprising the step of receiving physiological data about the user, and wherein the updating step is based at least in part on the received physiological data, preferably wherein the physiological data is received from a sensor of a wearable device.

9. (canceled)

10. The method of claim 1, wherein the information about the user is received from a wearable device or a consumption monitoring device.

11. The method of claim 1, wherein the step of generating a consumption threshold comprises the steps of analyzing at least two goals provided by the user, wherein the at least two goals will result in at least two different consumption thresholds, and determining the new threshold by selecting one of the at least two different consumption thresholds or making a compromise.

12. The method of claim 1, wherein the item is a consumable that affects the cardiovascular system, preferably wherein the item is coffee.

13. (canceled)

14. (canceled)

15. (canceled)

16. (canceled)

17. The method of claim 11, wherein the information about the user is at least one of, physiological information about the user, an activity of the user is scheduling information about the user.

18. (canceled)

19. (canceled)

20. The method of claim 11, wherein the step of updating the maximum and/or minimum consumption threshold comprises the step of prioritizing the received one or more goals from the user, and wherein the updating step utilizes the prioritized goals to update the maximum and/or minimum consumption threshold.

21. The method of claim 1, wherein:
the step of generating a consumption threshold is adapted so that the consumption threshold comprises a minimum consumption threshold and/or a maximum consumption threshold;
the step of updating the consumption threshold comprising updating the maximum and/or minimum consumption threshold.

22. A device for providing an item consumption threshold to a user, the device comprising:
a processor, wherein the processor is configured to: (i) receive one or more goals from the user; (ii) generate, based on the received one or more goals from the user, a maximum and/or minimum consumption threshold; (iii) receive information about the user; and update, based on the information about the user, the consumption threshold; and
a display, wherein the display is configured to provide the generated consumption threshold and updated consumption threshold to the user.

23. A system for providing an item consumption threshold to a user, the system comprising:
a consumption monitoring device configured to generate information about the user's consumption of the item, wherein the generated information is communicated to a processor, wherein the processor is configured to: (i) receive one or more goals from the user; (ii) generate, based on the received one or more goals from the user, a maximum and/or minimum consumption threshold; (iii) receive information about the user's consumption of the item; and update, based on the information about the user's consumption of the item, the consumption threshold; and
a display, wherein the display is configured to provide the generated consumption threshold and updated consumption threshold to the user.
